(19) United States
(12) Patent Application Publication
(10) Pub. No.: US 2012/0172255 A1
(43) Pub. Date: Jul. 5, 2012

(54) ES-MS OF GLYCOPEPTIDES FOR ANALYSIS OF GLYCOSYLATION

(75) Inventors: Markus Haberger, Muenchen (DE); Dietmar Reusch, Muenchen (DE); Maurice Selman, Gouda (NL); Manfred Wuhrer, Voorschoten (NL)

(73) Assignee: HOFFMANN-LA ROCHE, INC,, Nutley, NJ (US)

(21) Appl. No.: 13/394,788

(22) PCT Filed: Sep. 3, 2010
(86) PCT No.: PCT/EP2010/005437

§ 371 Date: Mar. 7, 2012

(30) Foreign Application Priority Data
Sep. 7, 2009 (EP) 09169616.1

(51) Int. Cl. C40B 30/10; C12Q 1/37

(52) U.S. Cl. 506/12; 435/23

(57) ABSTRACT

Herein is reported a method for the determination of the glycosylation of an immunoglobulin with electrospray mass spectrometry but without the need for a chromatographic purification step after the digestion of the immunoglobulin and prior to the mass spectrometric analysis.

Herein is reported a mass spectrometric method for the analysis of the glycosylation of an immunoglobulin which does not require a chromatographic separation step.

### BACKGROUND OF THE INVENTION

The glycosylation of a polypeptide is an important characteristic for many recombinantly produced therapeutic polypeptides. Glycosylated polypeptides, also termed glycoproteins, mediate many essential functions in eukaryotic organisms, e.g. humans, and some prokaryotes, including catalysis, signaling, cell-cell communication, activities of the immune system, molecular recognition and association. Glycoproteins account for the majority of non-cytosolic proteins in eukaryotic organisms (Lis, H., et al., Eur. J. Biochem. 218 (1993) 1-27). The introduction of the glycosylation is a cotranslational and posttranslational modification and, thus, is not genetically controlled. The biosynthesis of oligosaccharides is a multistep process involving several enzymes, which compete with each other for the substrate. Consequently, glycosylated polypeptides comprise a microheterogeneous array of oligosaccharides, giving rise to a set of different glycoforms containing the same amino acid backbone. Terminal sialylation of glycosylated polypeptides for example has been reported to increase serum-half life of therapeutics, and glycosylated polypeptides containing oligosaccharide structures with terminal galactose residues show increased clearance from circulation (Smith, P. L., et al., J. Biol. Chem. 268 (1993) 795-802). Thus, in the biotechnological production of therapeutic polypeptides, e. g. of immunoglobulins, the assessment of oligosaccharide microheterogeniety and its batch-to-batch consistency are important tasks.

Immunoglobulins differ significantly from other recombinant polypeptides in their glycosylation. Immunoglobulin G (IgG) e. g. is a symmetrical, multifunctional glycosylated polypeptide of an approximate molecular mass of 150 kDa. It is consisting of two identical Fab parts responsible for antigen binding and the Fc part responsible for effector function. Glycosylation tends to be highly conserved in IgG molecules at Asn-297, which is buried between the CH2 domains of the heavy chains, forming extensive contacts with the amino acid residues within the CH2 domain (Sutton, B. J. and Phillips, D. C., Biochem. Soc. Trans. 11 (1983) 130-132). The Asn-297 linked core oligosaccharide structures are heterogeneously processed, such that a specific IgG exists in multiple glycoforms. Variations exist in the site occupancy of the Asn-297 site (macroheterogeniety) or by variation in the oligosaccharide structure at the glycosylation site (microheterogeniety), see for example Jenkins, N., et al., Nature Biotechnol. 14 (1996) 975-981. Generally, the more abundant oligosaccharide groups in IgG mAb are asialo biantennary complex type glycans, primarily agalactosylated (G0), mono-galactosylated (G1), or bi-galactosylated (G2) types (Ghirlandaio, R., et al., Immunol. Lett. 68 (1999) 47-52).

Given the importance of glycosylation on functional properties of recombinant glycosylated polypeptides and the necessity of a well-defined and consistent product production process, an on-line or ad-line analysis of the glycosylation profile of recombinantly produced glycosylated polypeptides during the fermentation process is highly desirable.

Kuhlmann (Kuhlmann, F. E., et al., J. Am. Soc. Mass Spec. 6 (1995) 1221-1225) reported the post reverse-phase high-performance liquid chromatography column addition of a solution of 75% propionic acid and 25% 2-propanol in a ratio 1:2 to the column flow. High-performance liquid chromatography with electrospray ionization mass spectrometry (LCIMS) and liquid chromatography with tandem mass spectrometry (LC/MS/MS) were applied to the analysis of the site-specific carbohydrate heterogeneity in erythropoietin (EPO) (Kawasaki, N., et al., Anal. Biochem. 285 (2000) 82-91).

In U.S. 2006/0269979 a high throughput glycan analysis for diagnosing and monitoring rheumatoid arthritis and other autoimmune diseases is reported. An identification method of glycoproteins is reported in WO 2009/048196. In U.S. Pat. No. 7,351,540 protein isolation and analysis is reported. Development of an immunofluorometric assay for human kallikrein 15 is reported by Shaw et al. (Clin. Biochem. 40 (2007) 104-110).

### SUMMARY OF THE INVENTION

Herein is reported as one aspect a method for the determination of the glycosylation of an immunoglobulin comprising
enzymatically digesting the immunoglobulin,
absorbing the immunoglobulin fragments to Sepharose beads,
washing the Sepharose beads with the absorbed immunoglobulin fragments with a solution comprising trifluoroacetic acid,
recovering the immunoglobulin fragments from the Sepharose beads,
performing an electrospray mass spectrometry of the recovered immunoglobulin fragments, and
determining the glycosylation of the immunoglobulin from the mass spectrometric data.

### DETAILED DESCRIPTION OF THE INVENTION

The current invention is directed to a method for the determination of the glycosylation of an immunoglobulin with ES-MS without the need for a chromatographic purification step after the enzymatic digestion of the immunoglobulin and prior to the mass spectrometric analysis.

Human immunoglobulins are mainly glycosylated at the asparagine residue at position 297 (Asn297) with a core fucosylated biantennary complex oligosaccharide (numbering according to Kabat). Asn297 refers to the asparagine residue located at about position 297 in the Fc region (Eu numbering of Fc region residues) of an immunoglobulin. However, Asn297 may also be located about ±3 amino acids upstream or downstream of position 297, i.e., between positions 294 and 300, due to minor sequence variations occurring in immunoglobulins.

Immunoglobulins produced by mammalian cells typically comprise a branched, biantennary oligosaccharide that is generally attached by an N-linkage to Asn297 of the CH2 domain of the Fc region (see, e.g., Wright, A. and Morrison, S. L., Trend. Biotechnol. 15 (1997) 26-32). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. The biantennary glycostructure, i.e. the biantennary oligosaccharide, is terminated by up to two galactose residues in each arm. The arms are denoted (1,6) and (1,3) according to the bond to the central mannose residue. The glycostructure denoted as GO comprises no terminal galactose residue. The glycostructure denoted as G1 contains one or more galactose residues in one arm. The glycostructure denoted as G2 contains one or more galactose residues in each arm (Raju, T. S., Bioprocess Int. 1 (2003) 44-53). Human constant heavy chain regions are reported in detail by Kabat, E. A., et al., Sequences of Proteins of Immunological Interest, 5th Ed. Public Health Service, National Institutes of Health, Bethesda, Md. (1991); by Brueggemann, M., et al., J. Exp. Med. 166 (1987) 1351-1361; and by Love, T. W., et al., Methods Enzymol. 158 (1989) 515-527. CHO type glycosylation of antibody Fc parts is e.g. described by Routier, F. H., Glycoconjugate J. 14 (1997) 201-207.

The term “immunoglobulin” encompasses the various forms of immunoglobulins such as human immunoglobulins, humanized immunoglobulins, chimeric immunoglobulins, or T cell antigen depleted immunoglobulins (see e.g. WO 98/33523, WO 98/52976, and WO 00/34317). Genetic engineering of immunoglobulins is e.g. described in Morrison, S. L., et al., Proc. Natl. Acad Sci. USA 81 (1984) 6851-6855; U.S. Pat. No. 5,202,238 and U.S. Pat. No. 5,204,244; Riechmann, L., et al., Nature 332 (1988) 323-327; Neuberger, M. S., et al., Nature 314 (1985) 268-270; Lonberg, N., Nat. Biotechnol. 23 (2005) 1117-1125.

An immunoglobulin in general comprises two so called full length light chain polypeptides (light chain) and two so called full length heavy chain polypeptides (heavy chain). Each of the full length heavy and light chain polypeptides contains a variable domain (variable region) (generally the amino terminal portion of the full length polypeptide chain) comprising binding regions which can interact with an antigen. Each of the full length heavy and light chain polypeptides comprises a constant region (generally the carboxyl terminal portion). The constant region of the full length heavy chain mediates the binding of the antibody i) to cells bearing a Fc gamma receptor (FcγR), such as phagocytic cells, or ii) to cells bearing the neonatal Fc receptor (FcRn) also known as Brambell receptor. It also mediates the binding to some factors including factors of the classical complement system such as component (C1q). The variable domain of a full length immunoglobulin's light or heavy chain in turn comprises different segments, i.e. four framework regions (FR) and three hypervariable regions (CDR). A “full length antibody heavy chain” is a polypeptide consisting in N-terminal to C-terminal direction of an antibody heavy chain variable domain (VH), an antibody constant domain 1 (CH1), an antibody hinge region, an antibody constant domain 2 (CH2), an antibody constant domain 3 (CH3), and optionally an antibody constant domain 4 (CH4) in case of an antibody of the subclass IgE. A “full length antibody light chain” is a polypeptide consisting in N-terminal to C-terminal direction of an antibody light chain variable domain (VL), and an antibody light chain constant domain (CL). The full length antibody chains are linked together via inter-chain disulfide bonds between the CL-domain and the CH1 domain and between the hinge regions of the full length antibody heavy chains.

It has been reported in recent years that the glycosylation of immunoglobulins, i.e. the saccharide composition and multitude of attached glycostructures, has a strong influence on the biological properties (see e.g. Jefferis, R., Biotechnol. Prog. 21 (2005) 11-16). Immunoglobulins produced by mammalian cells contain 2-3% by mass oligosaccharides (Taniguchi, T., et al., Biochem. 24 (1985) 5551-5557). This is equivalent e.g. in an immunoglobulin of class G (IgG) to 2.3 oligosaccharide chains in an IgG of mouse origin (Mizuochi, T., et al., Arch. Biochem. Biophys. 257 (1987) 387-394) and to 2.8 oligosaccharide chains in an IgG of human origin (Parekh, R. B., et al., Nature 316 (1985) 452-457), whereof generally two are located in the Fc-region at Asn²⁹⁷ and the remaining in the variable region (Saba, J. A., et al., Anal. Biochem. 305 (2002) 16-31).

The term “glycosylation” denotes the sum of all oligosaccharides which are attached to all amino acid residues of an immunoglobulin. Due to the glycosylation heterogeneity of a cell, a recombinantly produced immunoglobulin comprises not only a single, defined N- or O-linked oligosaccharide at a specified amino acid residue, but is a mixture of polypeptides each having the same amino acid sequence but comprising differently composed oligosaccharides at the respective specified amino acid position. Thus, the above term denotes a group of oligosaccharides that are attached to specified amino acid positions of a recombinantly produced immunoglobulin, i.e. the heterogeneity of the attached oligosaccharide. The term “oligosaccharide” as used within this application denotes a polymeric saccharide comprising two or more covalently linked monosaccharide units.

For the notation of the different N- or O-linked oligosaccharides the individual sugar residues are listed from the non-reducing end to the reducing end of the oligosaccharide residue. The longest sugar chain was chosen as basic chain for the notation. The reducing end of an N- or O-linked oligosaccharide is the monosaccharide residue, which is directly bound to the amino acid of the amino acid backbone of the immunoglobulin, whereas the end of an N- or O-linked oligosaccharide, which is located at the opposite terminus as the reducing end of the basic chain, is termed non-reducing end.

An aspect as reported herein is a method for the determination of the glycosylation of an immunoglobulin comprising
enzymatically digesting the immunoglobulin,
absorbing the immunoglobulin fragments to Sepharose beads,
washing the Sepharose beads with the absorbed immunoglobulin fragments with a solution comprising trifluoroacetic acid,
recovering the immunoglobulin fragments from the Sepharose beads,
performing an electrospray mass spectrometry of the recovered immunoglobulin fragments, and
determining the glycosylation of the immunoglobulin from the mass spectrometric data.

It has been found that by washing the adsorbed immunoglobulin fragments with a solution comprising trifluoroacetic acid an improved electrospray mass spectrometric determination of the glycosylation of the immunoglobulin can be achieved. In one embodiment the concentration of the trifluoroacetic acid is of from 0.01% to 1% (v/v). In another embodiment the concentration of the trifluoroacetic acid is of from 0.05% to 0.5% (v/v). In still another embodiment the concentration of the trifluoroacetic acid is about 0.1% (v/v). Additionally a chromatographic purification step can be performed after the enzymatic digestion but is not necessary. As can be seen from the following Table 1 the washing with trifluoroacetic acid clearly improves the accuracy of the quantitative determination and concomitantly reduces the standard deviation (SD) and variation coefficient (VK) of the analysis results.

TABLE 1
Comparison of exemplary results for the determination of the
glycosylation of an exemplary anti-CCR5 antibody. The
determinations have been made in triplicate. The reference values
have been determined by ion exchange chromatography with pulsed
amperometric detection (Fuc = fucose).

| | | without trifluoroacetic | | | with trifluoroacetic acid | | |
|---|---|---|---|---|---|---|---|
| | | acid washing | | | washing | | |
| | reference | [%] | | | [%] | | |
| glyco- | value | | | variation | | | variation |
| structure | [%] | value | SD | coefficient | value | SD | coefficient |
| Man-Fuc | 28.4 | 23.4 | 0.6 | 2.7 | 22.4 | 0.3 | 1.3 |
| G(0)-Fuc | | 10.9 | 1.0 | 9.3 | 10.8 | 1.0 | 9.3 |
| G(0) | 44.3 | 40.1 | 3.0 | 7.5 | 43.4 | 0.4 | 1.0 |
| G(1) | 25.2 | 19.4 | 1.7 | 9.0 | 19.9 | 0.2 | 1.1 |
| G(2) | 2.1 | 6.2 | 3.8 | 61.1 | 3.4 | 0.7 | 21.9 |

The term “Sepharose” denotes a crosslinked form of agarose. Agarose is a linear polysaccharide comprising as monomeric building blocks agarobiose, which in turn is a disaccharide consisting of glycosidically linked D-galactose and 3,6-anhydro-L-galactopyranose.

In one embodiment the enzymatically digesting is by incubating with an enzyme selected from trypsin, chymotrypsin, papain, IdeS, and the endoproteinases Arg C, Lys C and Glu C. In another embodiment the enzymatically digesting is by incubating with trypsin.

It has further been found that it is advantageous to use a solution in the washing step with an acetonitrile concentration of from 78% to 88% (v/v). In one embodiment the acetonitrile concentration is of from 80% to 85% (v/v). In another embodiment the acetonitrile concentration is about 83% (v/v). The term “about” denotes that the thereafter following value is the center of a range of +/−10% of the value. Values beside that range have a negative influence on the quantitative determination. Therefore, in one embodiment the solution in the washing step comprises about 0.1% (v/v) trifluoroacetic acid and about 83% (v/v) acetonitrile. In one embodiment comprises the method the step of washing the Sepharose beads with a solution consisting of 78% to 88% (v/v) acetonitrile and water. In one embodiment the method comprises the step of washing the Sepharose beads with a solution consisting of 80% to 85% (v/v) acetonitrile and water. In one embodiment the washing is with a solution consisting of about 83% (v/v) acetonitrile and water. In a further embodiment the method comprises the step of adjusting the solution of the enzymatic digest to 78% to 88% (v/v) acetonitrile. In a further embodiment the method comprises the step of adjusting the solution of the enzymatic digest to 80% to 85% (v/v) acetonitrile. In one embodiment the adjusting is to about 83% (v/v) acetonitrile. In another embodiment the method comprises a second washing step with 78% to 88% (v/v) acetonitrile. In one embodiment the method comprises a second washing step with 80% to 85% (v/v) acetonitrile. In another embodiment the second washing is with about 83% (v/v) acetonitrile.

If a reference is made to a volumetric ratio (v/v) within this application the following applies:
depending on the intended final volume the relative volume of the acetonitrile fraction, e.g. 83%, is calculated from the intended final volume,
the calculated relative volume of acetonitrile is provided and water is added until the intended final volume is obtained,
thereafter the relative volume fraction of trifluoroacetic acid is added, calculated based on the intended final volume.

For example, one liter (1000 ml) of a solution consisting of 0.1% (v/v) trifluoroacetic acid, 83% (v/v) acetonitrile and water is obtained by providing 830 ml acetonitrile (83% of 1000 ml), adding water thereto until a volume of 1000 ml is reached, and thereafter adding 1 ml (0.1% (v/v) of 1000 ml) trifluoroacetic acid.

In one embodiment the method comprises as first step denaturating the immunoglobulin with a denaturing agent. In another embodiment the denaturing is at pH 8.5. In one embodiment the solution consists of 0.1% (v/v) trifluoro acetic acid, 83% (v/v) acetonitrile and water. In another embodiment the Sepharose beads are sepharose CL-4B beads. In one embodiment the applying to Sepharose beads is for 5 minutes.

In another embodiment the method comprises the step of washing the Sepharose beads with water. In this step the immunoglobulin fragments are recovered from the Sepharose beads.

It has further been found that without adding the solution containing 25%/75% (v/v) 2-propanol/propionic acid the ionization of the purified glycopeptides is very poor. Thus, although the method works without the addition it can be further improved by additionally adding a solution containing 25%/75% (v/v) 2-propanol/propionic acid. Additionally, the higher charge states of the glycopeptides, which can be used for a correct quantitation of the different glycopeptides species of glycopeptides, are increasingly present if a solution containing 25%/75% (v/v) 2-propanol/propionic acid is added. This can be seen for the fucosylated and G(2) forms from Table 2. Therefore, in one embodiment the method comprises the step of mixing the immunoglobulin fragments with a solution consisting of 25% (v/v) 2-propanol and 75% (v/v) propionic acid.

TABLE 2
Comparison of exemplary results for the determination of the
glycosylation of an exemplary anti-CCR5 antibody. The
determinations have been made in triplicate. The reference values
have been determined by ion exchange chromatography with pulsed
amperometric detection (Fuc = fucose).

| | | with trifluoroacetic | | | with trifluoroacetic | | |
|---|---|---|---|---|---|---|---|
| | | acid washing | | | acid washing | | |
| | | without addition of | | | with addition of 2-propanol | | |
| | | 2-propanol/propionic acid | | | and propionic acid | | |
| | reference | [%] | | | [%] | | |
| glyco- | value | | | variation | | | variation |
| structure | [%] | value | SD | coefficient | value | SD | coefficient |
| Man-Fuc | 28.4 | 22.4 | 0.3 | 1.3 | 22.2 | 0.0 | 0.0 |
| G(0)-Fuc | | 10.8 | 1.0 | 9.3 | 8.5 | 0.2 | 2.1 |
| G(0) | 44.3 | 43.4 | 0.4 | 1.0 | 44.3 | 0.8 | 1.8 |
| G(1) | 25.2 | 19.9 | 0.2 | 1.1 | 21.6 | 0.7 | 3.1 |
| G(2) | 2.1 | 3.4 | 0.7 | 21.9 | 3.5 | 0.3 | 8.4 |

| | | | | | | |
|---|---|---|---|---|---|---|
| Signal intensity | 3139.0 | 1326.4 | 42.3 | 7775.0 | 796.7 | 10.2 |
| [area units] | | | | | | |

The following examples and figures are provided to aid the understanding of the present invention, the true scope of which is set forth in the appended claims. It is understood that modifications can be made in the procedures set forth without departing from the spirit of the invention.

### DESCRIPTION OF THE FIGURE

FIG. 1 Schematic method diagram.

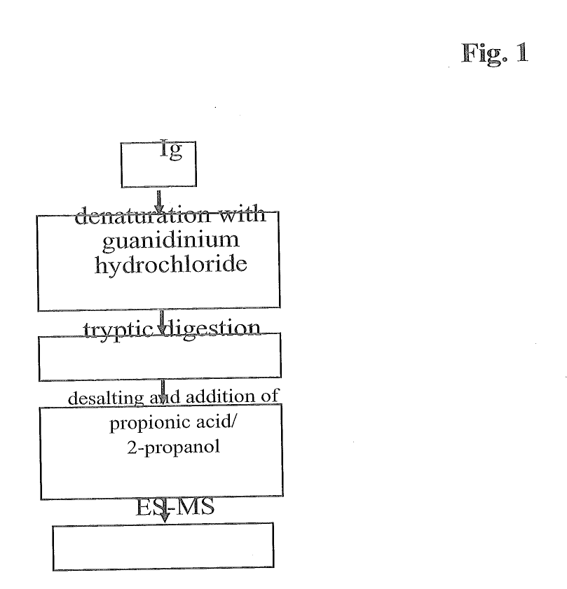

### MATERIALS

Tris (hydroxy aminomethane) hydrochloride (TRIS-HCl) and guanidinium-hydrochloride were purchased from Merck. Acetonitril (ACN), trifluoroacetic acid (TFA), hydrochloric acid, 2-propanol and propionic acid were obtained from VWR International Baker.

Trypsin was obtained from Roche Diagnostics GmbH, Mannheim, Germany. NAP5-Sephadex columns were obtained from GE Healthcare. CL-4B Sepharose beads were purchased form Amersham Bioscience. Multiscreen Solvinert 96 well 0.45 μm pore-size low-binding hydrophilic PTFE Filter Plates were obtained from Millipore.

The invention is exemplified with an anti-CCR5 antibody. The production thereof and the coding sequences thereof are reported e.g. in WO 2006/103100 and WO 2009/090032.

### EXAMPLE 1

### Digestion

300 μg of purified anti-CCR5 antibody were incubated for some minutes with guanidinium-hydrochloride at pH 8.5. After buffer exchange to TRIS-HCl pH 8.5 using a Sephadex column the antibody was digested without prior reduction with trypsin at 37° C. over night (16 hours).

### EXAMPLE 2

### Purification

1 ml of Sepharose CL-4B beads were washed three times with water. 15 μl of cleaned Sepharose beads were dissipated in 200 μl water and thereafter assigned to the wells of a 96-well Multiscreen filter plate. The beads were washed two times each with 200 μl of water and conditioned two times each with 200 μl of an 83% acetonitrile/water solution on a vacuum manifold using vacuum at <0.1 inch. Hg. 40 μl of the tryptic digest were adjusted to 83% (v/v) acetonitrile. The digest solution was thereafter applied to the conditioned Sepharose beads and incubated for five minutes with gentle shaking. The 96 well plate was covered with a suitable lid to prevent acetonitrile from evaporating. The beads were washed two times each with 200 μl 0.1% TFA-83% ACN (v/v) and two times each with 200 μl 83% (v/v) acetonitrile. During the washing steps the beads must be kept always wet to prevent the glycopeptides form eluting. The glycopeptides were recovered from the beads with three times 30 μl of water in a 96 well v-bottom plate.

### EXAMPLE 3

### Sample Preparation for Mass Spectrometry

For MS nanospray analysis the glycopeptides were mixed with 30 μl of a solution containing 25%/75% (v/v) 2-propanol/propionic acid. The prepared sample was directly infused to the mass spectrometer by means of a nanospray (NanoMate).

### EXAMPLE 4

### Mass Spectrometry

For the measurement a calibrated q-TOF Ultima from waters with a NanoMate source from Advion instead of the normal ultima nanospray source was used. 96 samples can be measured within 288 minutes completely automated.

1. Method for the determination of the glycosylation of an immunoglobulin comprising
enzymatically digesting the immunoglobulin,
absorbing the immunoglobulin fragments to Sepharose beads,
washing the Sepharose beads with the absorbed immunoglobulin fragments with a solution comprising trifluoroacetic acid,
recovering the immunoglobulin fragments from the Sepharose beads,
performing an electrospray mass spectrometry of the recovered immunoglobulin fragments, and
determining the glycosylation of the immunoglobulin from the mass spectrometric data.

2. Method according to claim 1, characterized in that the concentration of the trifluoroacetic acid in the washing step is of from 0.05% to 0.5% (v/v).

3. Method according to any one of the preceding claims, characterized in that the enzymatically digesting is by incubating the immunoglobulin in solution with an enzyme selected from trypsin, chymotrypsin, papain, IdeS, Arg C, Lys C and Glu C.

4. Method according to any one of the preceding claims, characterized in comprising the step of adjusting the solution of the enzymatic digest to 78% to 88% (v/v) acetonitrile.

5. Method according to any one of the preceding claims, characterized in comprising the step of absorbing the immunoglobulin fragments to the Sepharose beads in a solution comprising trifluoroacetic acid, 78% to 88% (v/v) acetonitrile and water.

6. Method according to any one of the preceding claims, characterized in comprising a second washing step of the sepharose beads with a solution consisting of 78% to 88% (v/v) acetonitrile and water.

7. Method according to any one of the preceding claims, characterized in comprising the step of recovering the immunoglobulin fragments by washing the Sepharose beads with water.

8. Method according to any one of the preceding claims, characterized in comprising after the recovering step the step of mixing the immunoglobulin fragments with a solution comprising 25% (v/v) 2-propanol and 75% (v/v) propionic acid.

9. Use of a method according to any one of the preceding claims in the analysis of the glycosylation of an immunoglobulin.

10. The use according to claim 9, characterized in that the analysis is an ad-line analysis or a high-throughput analysis.
